(19) United States
(12) Patent Application Publication
(10) Pub. No.: US 2025/0332444 A1
(43) Pub. Date: Oct. 30, 2025

(54) CANCER THERAPY DELIVERY SYSTEMS AND METHODS

(71) Applicant: Boston Scientific Scimed, Inc., Maple Grove, MN (US)

(72) Inventors: Patrick A. Haverkost, Crystal, MN (US); Joel N. Groff, Delano, MN (US); Scott McGhee, Ottawa (CA); David Raab, Roseville, MN (US); McKenzie Dudley, St. Louis Park, MN (US); Kianna Y. Elahi-Gedwillo, Minneapolis, MN (US)

(21) Appl. No.: 19/188,260

(22) Filed: Apr. 24, 2025

Related U.S. Application Data
(60) Provisional application No. 63/638,801, filed on Apr. 25, 2024.

(51) Int. Cl. A61N 5/10; A61K 51/12; A61M 5/14; A61M 5/145; A61M 5/158; A61M 5/168; A61M 5/36; A61M 39/20

(52) U.S. Cl. CPC A61N 5/1007; A61K 51/1255; A61M 5/1407; A61M 5/1413; A61M 5/1452; A61M 5/158; A61M 5/16877; A61M 5/36; A61M 39/20; A61K 2121/00; A61M 2005/1402; A61M 2206/20; A61M 2210/0693; A61N 2005/1021

(57) ABSTRACT

Embodiments herein relate to cancer therapy delivery systems with improved flow paths and features for preparing the same for use. In an embodiment, a cancer therapy delivery system is included having a carrier fluid delivery device and a first fluid line, wherein the first fluid line is in fluid communication with the carrier fluid delivery device. The system can also include a fluid injector and withdrawal assembly including an inflow conduit and an outflow conduit. The fluid injector and withdrawal assembly is in fluid communication with the first fluid line and a second fluid line. The system can also include a fluid delivery catheter in fluid communication with the second fluid line. The system can also include a jumper device configured to provide a direct fluid flow path between the inflow conduit and the outflow conduit. Other embodiments are also included herein.

This application claims the benefit of U.S. Provisional Application No. 63/638,801, filed Apr. 25, 2024, the content of which is herein incorporated by reference in its entirety.

### FIELD

Embodiments herein relate to cancer therapy systems. M ore specifically, embodiments herein relate to cancer therapy delivery systems with improved flow paths and features for preparing the same for use.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Chemotherapy and immunotherapy are other common therapeutic approaches but can include substantial side effects. The use of radiation represents another approach. Specifically, radiation therapy aims at damaging the DNA of cancer cells so that they lose the capability to divide and proliferate, thus leading to the cell death process for the cancerous cells.

### SUMMARY

Embodiments herein relate to cancer therapy delivery systems with improved flow paths and features for preparing the same for use. In a first aspect, a cancer therapy delivery system can be included having a carrier fluid delivery device, a first fluid line in fluid communication with the carrier fluid delivery device, and a fluid injector and withdrawal assembly. The fluid injector and withdrawal assembly can include an inflow conduit and an outflow conduit. The fluid injector and withdrawal assembly can be in fluid communication with the first fluid line. A second fluid line in fluid communication with the outflow conduit can also be included. Further, a removable jumper device can be included that is configured to provide a direct fluid flow path between the inflow conduit and the outflow conduit.

In a second aspect, in addition to one or more of the preceding or following aspects, or in the alternative to some aspects, the carrier fluid delivery device can include a syringe assembly.

In a third aspect, in addition to one or more of the preceding or following aspects, or in the alternative to some aspects, the inflow conduit can include a first needle and the outflow conduit can include a second needle.

In a fourth aspect, in addition to one or more of the preceding or following aspects, or in the alternative to some aspects, the jumper device fits over a bottom end of the first needle and a bottom end of the second needle when the needles are in a deployed position.

In a fifth aspect, in addition to one or more of the preceding or following aspects, or in the alternative to some aspects, the jumper device can include a cap.

In a sixth aspect, in addition to one or more of the preceding or following aspects, or in the alternative to some aspects, the jumper device can include a cap configured to be attached to a bottom end of the fluid injector and withdrawal assembly.

In a seventh aspect, in addition to one or more of the preceding or following aspects, or in the alternative to some aspects, the cap can be configured to be attached to a bottom end of the fluid injector and withdrawal assembly using a pressure-fit mechanism.

In an eighth aspect, in addition to one or more of the preceding or following aspects, or in the alternative to some aspects, the cap can define an internal volume serving as a flow path.

In a ninth aspect, in addition to one or more of the preceding or following aspects, or in the alternative to some aspects, the internal volume serving as a flow path can have a volume of less than 5 mL.

In a tenth aspect, in addition to one or more of the preceding or following aspects, or in the alternative to some aspects, the cap can define an internal passageway that serves as a flow path.

In an eleventh aspect, in addition to one or more of the preceding or following aspects, or in the alternative to some aspects, the system can further include a deployment restriction device, wherein the deployment restriction device can be removably attached to the fluid injector and withdrawal assembly, and wherein the deployment restriction device can be configured to limit vertical travel of the inflow conduit and the outflow conduit.

In a twelfth aspect, in addition to one or more of the preceding or following aspects, or in the alternative to some aspects, the cancer therapy delivery system can further include a first deployment lever configured to move vertically along the fluid injector and withdrawal assembly causing vertical movement of the inflow conduit. The system can further include a second deployment lever, wherein the second deployment lever can be configured to move vertically along the fluid injector and withdrawal assembly causing vertical movement of the outflow conduit.

In a thirteenth aspect, in addition to one or more of the preceding or following aspects, or in the alternative to some aspects, the first deployment lever and the second deployment lever can be configured to slide vertically within slots defined by a body member of the fluid injector and withdrawal assembly.

In a fourteenth aspect, in addition to one or more of the preceding or following aspects, or in the alternative to some aspects, the jumper device can be configured to be removed before the fluid injector and withdrawal assembly can be fitted into a mixing chamber.

In a fifteenth aspect, in addition to one or more of the preceding or following aspects, or in the alternative to some aspects, one or more of the outflow conduit and the second fluid line can have an inner diameter less than an inner diameter of one or more of the inflow conduit, and the first fluid line.

In a sixteenth aspect, a method of delivering cancer therapy can be included. The method can include preparing a cancer therapy delivery system for use. Preparing the system for use can include pushing a fluid from a carrier fluid delivery device through a first fluid line, a fluid injector and withdrawal assembly, a flow jumper spanning portions of the fluid injector and withdrawal assembly, and a second fluid line, and removing air bubbles from the system. The method can further include removing the flow jumper, connecting the fluid injector and withdrawal assembly with a mixing chamber, and pushing a carrier fluid through the system with the carrier fluid delivery device causing the carrier fluid to move through the system and through the fluid injector and withdrawal assembly into the mixing chamber to form a suspension or mixture of radioactive therapeutic microspheres and the carrier fluid and out of the fluid injector and withdrawal assembly and through a fluid line and into a microcatheter.

In a seventeenth aspect, in addition to one or more of the preceding or following aspects, or in the alternative to some aspects, the flow jumper can be configured to provide a direct fluid flow path between an inflow conduit and an outflow conduit of the fluid injector and withdrawal assembly.

In an eighteenth aspect, in addition to one or more of the preceding or following aspects, or in the alternative to some aspects, the radioactive therapeutic microspheres have an average diameter of about 20 μm to about 30 μm.

In a nineteenth aspect, in addition to one or more of the preceding or following aspects, or in the alternative to some aspects, the method of delivering cancer therapy can be applied to treat a brain tumor.

In a twentieth aspect, in addition to one or more of the preceding or following aspects, or in the alternative to some aspects, the method of delivering cancer therapy can be applied to treat glioblastoma.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cancer therapy delivery system in accordance with various embodiments herein.

FIG. 2 is a partial sectional view of some components of a cancer therapy delivery system in accordance with various embodiments herein.

FIG. 3 is a sectional view of some components of a cancer therapy delivery system in accordance with various embodiments herein.

FIG. 4 is a perspective view of some components of a cancer therapy delivery system in accordance with various embodiments herein.

FIG. 5 is a schematic view of some components of a cancer therapy delivery system in accordance with various embodiments herein.

FIG. 6 is a sectional view of a jumper device in accordance with various embodiments herein.

FIG. 7 is a sectional view of a jumper device in accordance with various embodiments herein.

FIG. 8 is a schematic view of a cancer therapy delivery system in accordance with various embodiments herein.

FIG. 9 is a flow chart of operations of a method of delivering cancer therapy in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Radiation therapy aims at damaging the DNA of cancer cells so that they lose the capability to divide and proliferate, thus leading to the cell death process for the cancerous cells. Brachytherapy is a form of radiation therapy where a sealed radiation source is placed inside or next to the area requiring treatment. As one form of brachytherapy, targeted radioembolization therapy can be used to treat unresectable tumors. For example, Y-90 glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy.

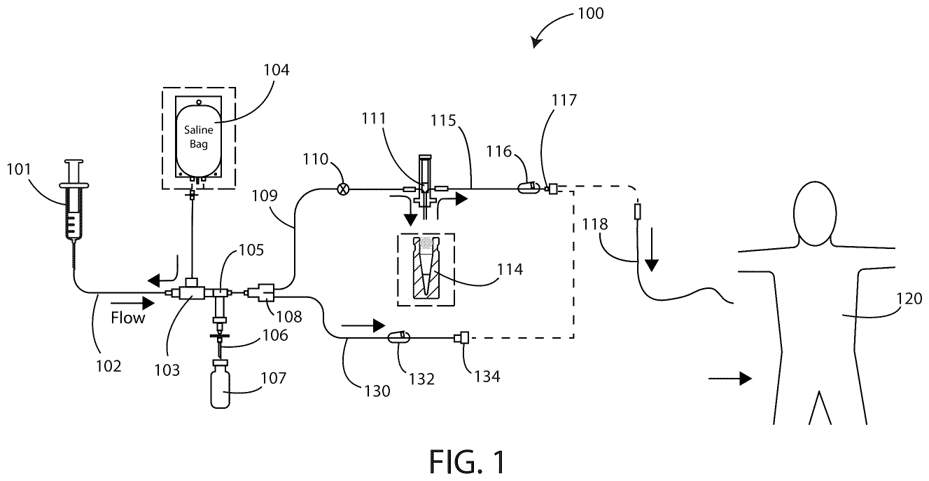

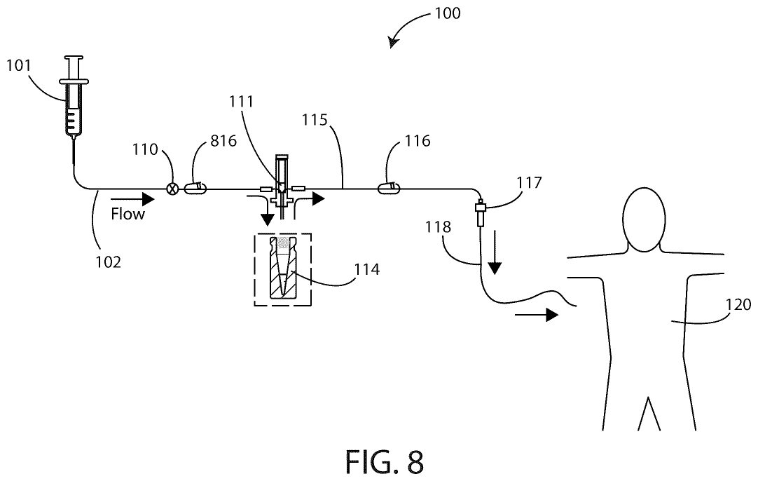

M any preparatory steps must be taken before the system is ready to deliver the radioactive microspheres to the patient including various assembly steps, priming, bubble removal, etc. However, embodiments herein include cancer therapy systems with improved flow paths and features for preparing the same for use. These features can make it easier to prepare the system for use, saving valuable time. For example, in some embodiments, a jumper device can be included that, as described more fully below, can provide a direct fluid flow path between certain elements of the system to make the priming operations faster and easier. Further, in some embodiments, the overall set of components used with the system can be reduced (such by comparison with the setup illustrated in FIG. 1—as described below with reference to FIG. 8), further easing preparatory operations and use of the system.

Referring now to FIG. 1, a schematic diagram is shown of components of an exemplary cancer-therapy delivery system 100 in accordance with various embodiments herein. Major parts of the cancer therapy delivery system 100 include a therapeutic fluid delivery device 101 (which in some cases can take the form of a syringe or syringe-like device), a fluid supply tube or line 102, and a dual check valve 103. In this example, the cancer-therapy delivery system 100 also includes a saline supply reservoir 104 (or fluid reservoir). The saline can serve as a carrier fluid to be mixed with the microspheres. The saline solution can be at various concentrations such as (0.3%, 0.5%, 0.7%, 0.9%, or the like). In some embodiments, the carrier fluid (typically a saline solution) can also include one or more other components. For example, in some embodiments the carrier fluid can include heparin (in the case of saline, a heparinized saline solution).

The system 100 can also include pressure relief valve 105, vented spike 106, overflow vial 107, “Y” fitting 108, fluid line 109, and check valve 110. The cancer-therapy delivery system 100 also includes a fluid injector and withdrawal assembly 111 along with a radioactive microsphere supply reservoir/mixing chamber assembly 114. The system 100 can also include outflow line 115, pinch clamp 116 and outflow connector 117.

The cancer-therapy delivery system 100 can also include and/or be connected to a microcatheter 118. FIG. 1 also shows a patient 120 into which the microcatheter 118 can be inserted to deliver the therapeutic suspension of microspheres. In some embodiments, the microcatheter 118 can specifically be one with a relatively small diameter, such as a microcatheter with a neuro use indication (hereafter “neurocatheter”). In some embodiments, the microcatheter 118 diameter can be as small as 0.33 millimeters (mm) (0.013 inches), or even less. However, in other embodiments the catheter or microcatheter can be larger in diameter. In some embodiments, the catheter or microcatheter can have a diameter of less than or equal to 7.31, 5.33, 4.32, 4.01, 3.66, 3.33, 3.00, 2.67, 2.34, 2.01, 1,68, 1.35, 0.99, 0.66, or even 0.33 mm (0.288, 0.21, 0.17, 0.158, 0.144, 0.131, 0.118, 0.105, 0.092, 0.079, 0.066, 0.053, 0.039, 0.026, or even 0.013 inches (equivalent to 1 Fr)), or a diameter falling within a range between any of the foregoing.

While not intending to be bound by theory, inner diameters greater than a certain point can lead to undesirable microsphere dropout. As such, in various embodiments herein, the inner diameter of the microcatheter (or the inner diameter of a fluid passage within the catheter) can be quite small. For example, in some embodiments, the microcatheter 118 inner diameter can be less than or equal to 0.050, 0.045, 0.040, 0.035, 0.030, 0.035, 0.020, or 0.015 inches (1.27, 1.143, 1.016, 0.889, 0.762, 0.508, or 0.381 mm), or a size falling within a range between any of the foregoing.

In use, various operations can be performed to prepare the system 100. For example, operations can be formed such as assembly, system priming, air/bubble removal, and the like. In the context of the system configuration of FIG. 1, additional components can be utilized during such preparatory operations. By way of example, priming line 130, pinch clamp 132, and priming line connector (or Luer) 134 can be utilized. In some preparatory operations (e.g., priming, bubble removal, etc.), the priming line 130 (and specifically the priming line connector 134) can be connected to outflow connector 117. Then, a fluid can be pulled in from saline supply reservoir 104 and then pushed through the system using the therapeutic fluid delivery device 101, including first pulling in fluid from saline supply reservoir 104, causing fluid (such as saline) to be withdrawn from the saline supply reservoir 104. Then, with fluid injector and withdrawal assembly 111 not connected to mixing chamber assembly 114, the fluid can flow through fluid delivery device 101, dual check valve 103, pressure relief valve 105, before reaching “Y” fitting 108. At that point the fluid can follow one path through check valve 110 and into fluid injector and withdrawal assembly 111. The fluid can also follow another path through priming line 130, pinch clamp 132, and priming line connector 134, entering the other side of fluid injector and withdrawal assembly 111. The fluid can then pass out of fluid injector and withdrawal assembly 111 through needles or conduits thereof described below. In this way, both sides (inflow and outflow) of the fluid injector and withdrawal assembly 111 can be primed. This can be performed until all bubbles are removed from the system.

In general, priming operations are performed before introducing the fluid injector and withdrawal assembly 111 to the dose vial to ensure that air is not introduced to the patient when starting to flush through the dose vial to the catheter. In addition, priming that includes passing fluid through the dose vial would risk moving the microspheres before priming is complete and the patient is ready to receive the microspheres. As such, after priming operations are complete, then connector 134 can be disconnected from the downstream side of fluid injector and withdrawal assembly 111. Further, the fluid injector and withdrawal assembly 111 can be connected to the mixing chamber assembly 114 and the outflow connector 117 can be connected to the microcatheter 118.

Then (omitting some possible operations for ease of explanation) the clinician or other system user can pull back on a plunger or similar mechanism of therapeutic fluid delivery device 101 causing fluid (such as a saline solution) to be withdrawn from the saline supply reservoir 104, through the dual check valve 103 and the fluid supply tube 104, and into the fluid delivery device 101. Then the clinician or other system user can depress the plunger causing fluid to flow from the therapeutic fluid delivery device 101, through the fluid supply tube 102, through the dual check valve 103, pressure relief valve 105, “Y” fitting 108, check valve 110, and into the fluid injector and withdrawal assembly 111.

The fluid injector and withdrawal assembly 111 can be in fluid communication with the mixing chamber assembly 114 and can direct a flow of fluid into the mixing chamber assembly 114 coming from the therapeutic fluid delivery device 101 or pump such as through one of a pair of needles, cannulas, or tubes serving as an inflow conduit. The fluid can become mixed with microspheres in the mixing chamber assembly 114 forming a suspension which can then exit via the fluid injector and withdrawal assembly 111 via another needle, cannula, or tube serving as an outflow conduit and through outflow line 115, pinch clamp 116, and out of outflow connector 117 and into the microcatheter 118 and then into a desired site of the patient 120. After an initial volume of fluid is passed through to the patient this way, one or more flushes can be performed (e.g., additional amounts of carrier fluid can be run through the system and to the patient to ensure that all or nearly all of the microspheres are delivered to the patient).

The microspheres must be incorporated into a mixture or suspension with a carrier fluid and then withdrawn from a dose vial within the mixing chamber assembly before they can be delivered to the patient. Before this can happen, however, the fluid injector and withdrawal assembly must be connected to the mixing chamber assembly.

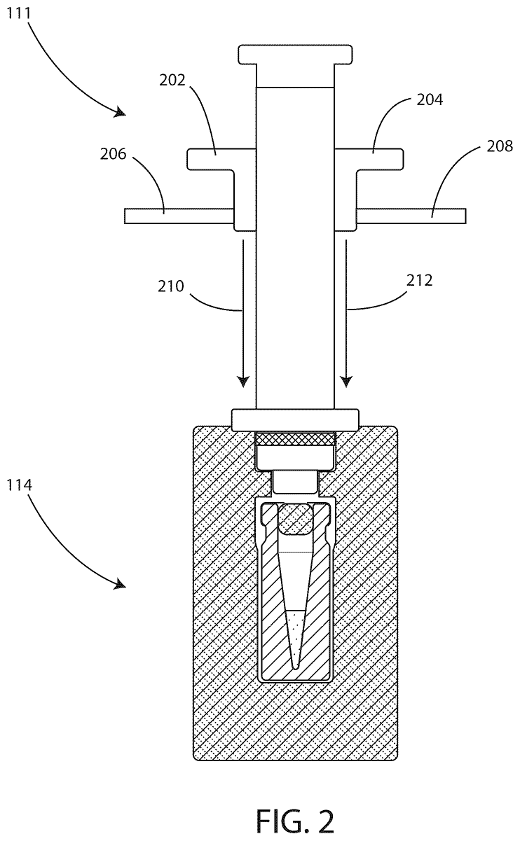

Referring now to FIG. 2, a partial sectional view is shown of some components of a cancer therapy delivery system in accordance with various embodiments herein. In specific, FIG. 2 shows a fluid injector and withdrawal assembly 111 engaged with the mixing chamber assembly 114. The fluid injector and withdrawal assembly 111 is shown with inflow port 206, through which a carrier fluid is brought in. The fluid injector and withdrawal assembly 111 also includes outflow port 208, through which a mixture or suspension of the carrier fluid and the therapeutic microspheres is carried out.

The formation of the mixture or suspension is carried out in the mixing chamber assembly 114. However, in an initial state, the needles or similar elements of the withdrawal assembly 111 that pass into the vial are in an at least partially raised or retracted position. They must be lowered to pass into the vial of the mixing chamber assembly 114 before the microspheres can be delivered to the patient. For this purpose, the cancer therapy delivery system can include a first deployment lever or tab 202, which can undergo vertical travel 210. Further, the cancer therapy delivery system can also include a second deployment lever or tab 204, which can undergo vertical travel 212. The deployment levers 202 and 204 can be operatively connected to the needles or similar structures and can cause them to be lowered when the deployment levers are lowered. In some embodiments, the deployment levers can be connected internally or otherwise linked so that movement of the deployment levers happens together. However, in some embodiments, movement of the needle or similar structure on the inflow side can be controlled separately from movement of the need or similar structure on the outflow side.

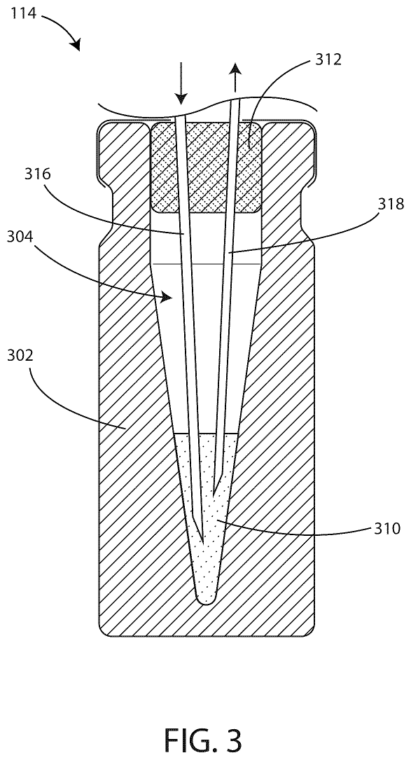

Referring now to FIG. 3, a sectional view of some components of a cancer therapy delivery system is shown in accordance with various embodiments herein. In specific, a mixing chamber assembly 114 is shown along with an inflow conduit 316 (or needle) and an outflow conduit 318 (or needle) in a lowered or fully deployed position wherein they have pierced a septum 312 and passed into a vial 302, and specifically, an internal volume 304 defined therein. Radioactive glass microspheres 310 (such as described in further detail below) can be disposed within the internal volume 304. In use, a carrier fluid enters the internal volume through the inflow conduit 316 or needle and mixes within the radioactive glass microspheres 310 therein creating a mixture or suspension. Then the mixture or suspension passes out of the internal volume 304 through the outflow conduit 318. In some embodiments, the flow rate of fluid into and out of the internal volume 304 can be less than or equal to about 25, 20, 25, 10, 8, 6, or 5 milliliters/minute (mL/min), or a flow rate falling within a range between any of the foregoing. For example, in some embodiments, the flow rate can be from 5 to 20 mL/min.

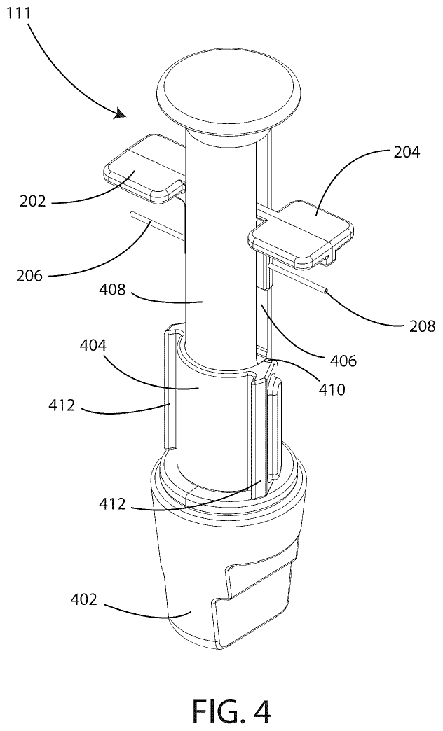

The systems herein can include various features to facilitate preparatory operations that must be performed before the system is ready to deliver radioactive microspheres. Referring now to FIG. 4, a perspective view of some components of a cancer therapy delivery system is shown in accordance with various embodiments herein. In specific, FIG. 4 shows a fluid injector and withdrawal assembly 111. As before, the fluid injector and withdrawal assembly 111 includes an inflow port 206 and an outflow port 208. The cancer therapy delivery system also includes a first deployment tab or lever 202 and a second deployment tab or lever 204. The fluid injector and withdrawal assembly 111 also includes a slot 406 defined by a body member 408 thereof. The second deployment lever 204 can slide vertically within the slot 406. A similar slot can be disposed on the other side of the fluid injection and withdrawal assembly 111 to facilitate vertical movement of the first deployment lever 202.

In various embodiments herein, the cancer therapy delivery system can also include a jumper device 402. The jumper device 402 can be configured to provide a fluid flow path between an inflow conduit and an outflow conduit of the fluid injector and withdrawal assembly 111, such as during priming operations. In various embodiments, the jumper device 402 fits over a bottom end of a first needle and a bottom end of a second needle when the needles are in a priming position (or partially deployed position). After priming operations and/or other preparatory operations are performed, then the jumper device 402 can be removed. As such, in various embodiments, the jumper device 402 can be configured to be removed before a fluid injector and withdrawal assembly 111 is fitted into a mixing chamber assembly 114. The jumper device 402 can be removable by hand. The jumper device 402 can be formed of various materials, such as biocompatible materials and, at least in some cases, sterilizable materials. By way of example, materials can include various polymers, glasses, ceramics, metals, composites, and the like. In some embodiments, the jumper device 402 can be formed of an elastomer or elastomeric polymer. Exemplary materials can specifically include a silicone rubber (an elastomeric polysiloxane polymer), ethylene diene monomer rubber (or EPDM rubber), and the like.

In this embodiment, the cancer therapy delivery system also includes a movement or deployment restriction device 404. In various embodiments, the deployment restriction device 404 can take the form of a clip or other structure and can be removably attached to a fluid injector and withdrawal assembly 111. In various embodiments, the deployment restriction device 404 can be configured to limit vertical travel of an inflow conduit (or needle) 316 and an outflow conduit (or needle) 318 by physically blocking movement of deployment levers 202 and 204 downward past a certain point preventing full deployment of the conduits or needles until the deployment restriction device 404 is removed.

In some embodiments, the deployment restriction device 404 can at least partially wrap around a portion of the body member 408 of the fluid injector and withdrawal assembly 111. In some embodiments, the deployment restriction device 404 can include a projection 410, tooth, or other structure to engage with a portion of the body member 408, such as the slots 406 thereof and thereby hold the deployment restriction device 404 in place. In some embodiments, the deployment restriction device 404 can include manipulation grips 412. In use, a system user can grasp manipulation grips 412 and, for example, pinch the two at least partially together which can facilitate removal of the deployment restriction device 404 from the fluid injector and withdrawal assembly 111. Removal of the deployment restriction device 404 can allow deployment lever 202 and deployment lever 204 to move vertically to transition from a priming position downward to a fully deployed position. Such movement will be described in greater detail below.

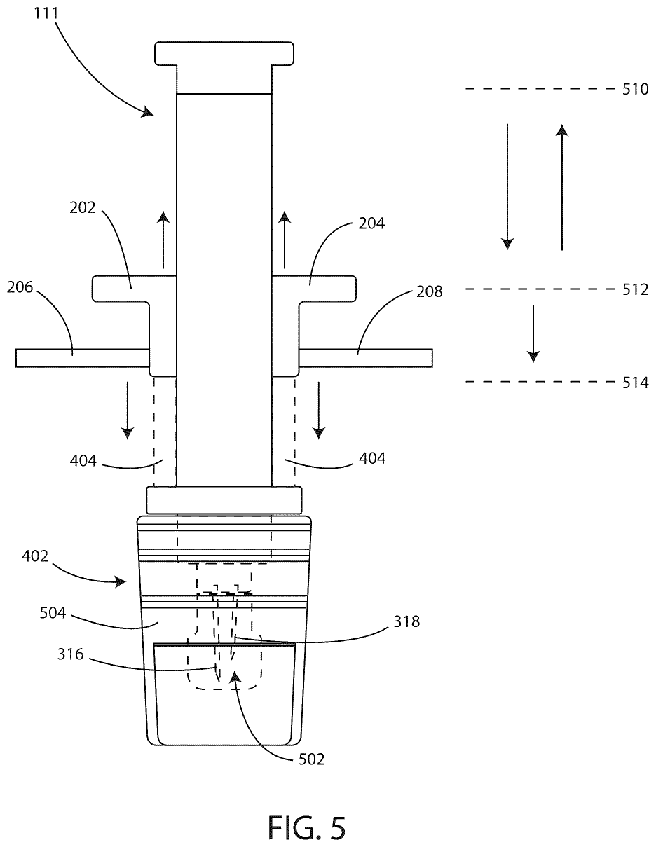

Referring now to FIG. 5, a schematic view of some components of a cancer therapy delivery system is shown in accordance with various embodiments herein. A s before, the fluid injector and withdrawal assembly 111 includes inflow port 206, outflow port 208, an inflow conduit 316 (or needle), an outflow conduit 318 (or needle), a first deployment tab or lever 202, a second deployment tab or lever 204, and a jumper device 402. As can be seen in this view, the jumper device 402 fits over a bottom end of the inflow conduit 316 and a bottom end of the outflow conduit 318 when the conduits (or needles) are in an intermediate position for system priming, such as shown in FIG. 5.

The deployment tabs or levers (202, 204) can move between a fully retracted position 510 wherein the conduits or needles are fully retracted within the fluid injector and withdrawal assembly 111 and other positions. For example, the deployment tabs or levers (202, 204) can also be moved to a priming position 512, in which the conduits or needles extend out from the fluid injector and withdrawal assembly 111 to be positioned within the internal volume 502 of the jumper device 402, such as shown in FIG. 5 for priming operations. The deployment tabs or levers (202, 204) can also be moved to a fully deployed position 514, such as after the fluid injector and withdrawal assembly 111 engages with mixing chamber assembly 114 so that the conduits or needles can extend into the internal volume 304 of the vial 302 for generation of the mixture or suspension and delivery of the same. In various embodiments, a locking mechanism (such as a snap-lock element or a detent mechanism) can be included so that once the deployment tabs or levers (202, 204) are moved to a fully deployed position they cannot be raised again (e.g., cannot revert back to the priming position 512 or the fully retracted position 510).

The jumper device 402 includes a cap 504 in this embodiment. The cap 504 can be formed of various materials including, for example, various polymers or composites, or even metals and glasses. In some embodiments, the cap 504 can be injection molded or manufactured using additive manufacturing techniques. The cap 504 can define an internal volume 502, which can serve to receive the ends or tips of the inflow conduit 316 and the outflow conduit 318. The internal volume 502 can also serve as a flow path to convey fluid from the tip of the inflow conduit 316 directly to the tip of the outflow conduit 318. This flow path can greatly simplify preparatory operations such as priming of the system. In various embodiments, the internal volume 502 can also serve as a bubble trap, such that fluid with an air bubble coming into the internal volume 502 from the inflow conduit 316 would have the bubble caught in the internal volume 502 such that it does not then pass into the outflow conduit 318. In various embodiments, the internal volume 502 can be relatively small. In some embodiments, the internal volume 502 serving as a flow path can have a volume of less than 10, 5, 4, 3, 2, or even 1 mL, or a volume falling within a range between any of the foregoing.

The cap 504 can be configured to be removably attached to a bottom end of the fluid injector and withdrawal assembly 111. For example, in some embodiments the cap 504 can be configured to be removably attached to a bottom end of the fluid injector and withdrawal assembly 111 with a pressure-fit or friction-fit mechanism. However, in other embodiments, the cap 504 can be configured to be screwed onto a bottom end of the fluid injector and withdrawal assembly 111 or can be attached using other mechanical or non-mechanical approaches.

It will be appreciated that jumper devices herein can take various forms and, specifically, the shape of the internal volume 502 used for transferring fluid from the inflow conduit to the outflow conduit 318 can take on many different shapes.

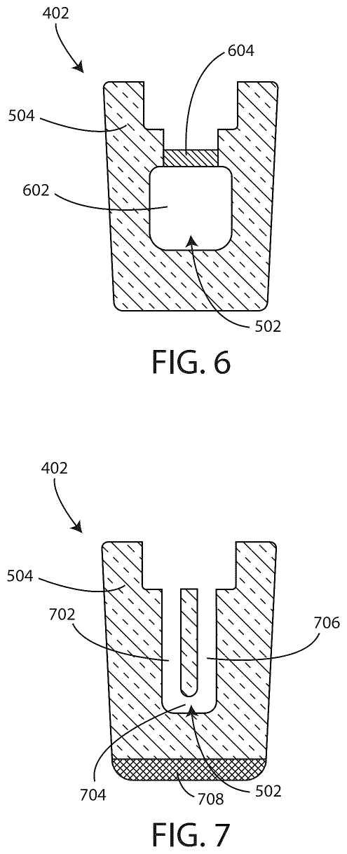

Referring now to FIG. 6, a sectional view of a jumper device is shown in accordance with various embodiments herein. As before, the jumper device 402 can include an internal volume 502 and a cap 504. In this example, the internal volume 502 includes a single chamber 602. The single chamber 602 can receive the ends or tips of the inflow conduit 316 and the outflow conduit 318 and serve as a flow path to convey fluid from the tip of the inflow conduit 316 directly to the tip of the outflow conduit 318. In some embodiments, a septum 604 can be disposed within the jumper device. The ends or tips of the inflow conduit 316 and the outflow conduit 318 can pierce the septum 604 and the septum 604.

However, in some scenarios it can be advantageous to minimize the volume within the jumper device. For example, this can allow priming to occur with a reduced volume of fluid. One approach to achieving this can be to include a septum as shown in FIG. 6. However, another approach to achieving this can include breaking up the internal volume to include channels for each of the inflow conduit and the outflow conduit as well as an internal passageway in between. As an example of this approach, referring now to FIG. 7, a sectional view of a jumper device is shown in accordance with various embodiments herein. In this embodiment, the cap 504 defines a first chamber 702, an internal passageway 704 (such as an internal channel, passage, conduit, or the like), and a second chamber 706. The first chamber 702 can receive the inflow conduit (or needle) and the second chamber 706 can receive the outflow conduit (or needle). This can be effective to reduce the volume within the cap and therefore reduce the amount of fluid required to fill the same during priming operations. The total volume of the first chamber 702, the internal passageway 704, and the second chamber 706 can be less than, for example, the comparable single chamber embodiments as shown in FIG. 6.

In some embodiments, the cap 504 can include a needle-impenetrable layer 708 attached to a bottom end thereof (or positioned elsewhere on or in the bottom side of cap 504). In this manner, even when the cap 504 is formed of a material (such as an elastomer or other polymer) that may be able to be penetrated by sharp ends of inflow conduit 316 and outflow conduit 318, the cap can still prevent penetration of such sharp ends through the bottom end of cap 504 for safety purposes. The needle-impenetrable layer 708 can be formed of various materials such as a rigid polymer, a metal, a composite, a glass or ceramic, or the like.

In some embodiments, operations can be simplified and/or operations can be made easier to execute (e.g., operations such as system assembly, priming of the system, removing bubbles therefrom, delivering therapy, flushing of the system, etc.) by reducing the number of components of the system. Referring now to FIG. 8, a schematic view of a cancer therapy delivery system 100 is shown in accordance with various embodiments herein with a reduced set of components as compared with that shown in FIG. 1.

In this example, the cancer therapy delivery system 100 includes a fluid delivery device 101, which pushes a carrier fluid through fluid supply tube 102, and a check valve 110. In some embodiments, the system 100 can also include an upstream pinch clamp 816. The carrier fluid then passes through to fluid injector and withdrawal assembly 111. During preparatory operations (priming, etc.) the fluid can pass through a jumper device as described above and onto components on the downstream side of fluid injector and withdrawal assembly 111. During delivery of therapy, the fluid can pass through fluid injector and withdrawal assembly 111 and then through the mixing chamber assembly 114. After leaving the mixing chamber assembly 114 and the fluid injector and withdrawal assembly 111, the mixture or suspension of radioactive microspheres and carrier fluid can pass through outflow line 115, downstream pinch clamp 116, and outflow connector 117 (or outlet Luer), before entering microcatheter 118, and patient 120. In some embodiments, an element such as a cap can be used to engage the outflow connector 117 when not actively priming to maintain sterility before connection the microcatheter 118. Engagement of both the upstream pinch clamp 816 and the downstream pinch claim 116 can result in fluid lock around the fluid injector and withdrawal assembly 111, such as to prevent any air from entering the same after priming and before therapy delivery.

However, in contrast with the configuration shown in FIG. 1, dual check valve 103, saline supply reservoir 104, pressure relief valve 105, vented spike 106, overflow vial 107, “Y” fitting 108, priming line 130, pinch clamp 132, and then connector 134 can all be omitted. In some cases, fluid delivery device 101 can be configured to utilize multiple containers or cartridges of carrier fluid that can be fitted into fluid delivery device 101 instead of withdrawing fluid saline supply reservoir 104.

Taken together, these changes can greatly simplify operations, for example, by reducing the number of steps that must be taken during system preparation, providing fewer junctures or connections between components thereby providing few places that may otherwise hold onto bubbles. These changes can also reduce the total volume of fluid needed for priming/flushing operations. In addition, removal of some components, like pressure relief valve 105, can advantageously make the system suitable for used with small diameter catheters, such as a neurocatheter. For example, the system can be suitable for use with microcatheters as small as 0.33 mm (0.013 inches) in diameter. Furthermore, configurations such as that shown in FIG. 8 can allow check valve 110 to be moved closer to fluid delivery device 101, such as within a line or flow distance of less than 15, 12.5, 10, 7.5, 5, 4, 3, or even 2 centimeters (5.91, 4.92, 3.93, 2.95, 1.97, 1.57, 1.18, or even 0.78 inches), or a distance falling within a range between any of the foregoing.

In some embodiments, one or more components of the system from the point of leaving the vial holding the microspheres can have a reduced inner diameter as compared with components upstream of the vial. By way of example, some components of the system from the point of leaving the vial can have an inner diameter of less than or equal to 0.89, 0.76, 0.64, or even 0.51 mm (0.035, 0.030, 0.025, or even 0.020 inches), or an inner diameter falling within a range between any of the foregoing. For example, one or more of the outflow conduit (or needle) 318, outflow port 208, and outflow line 115 can have such a reduced diameter. While not intending to be bound by theory, it is believed that this can lead to reduced retention of microspheres within the system downstream of the vial. In contrast, some components herein that are upstream of the vial can have a somewhat larger diameter or can be substantially the same. In some embodiments, some components of the system that are upstream of the vial can have an inner diameter of 1.02, 1.14, 1.27 mm (0.040, 0.045, 0.050 inches) or more, or an inner diameter falling within a range between any of the foregoing.

### Methods

Many different methods are contemplated herein, including, but not limited to, methods of making, methods of using, and the like. Aspects of system/device operation described elsewhere herein can be performed as operations of one or more methods in accordance with various embodiments herein.

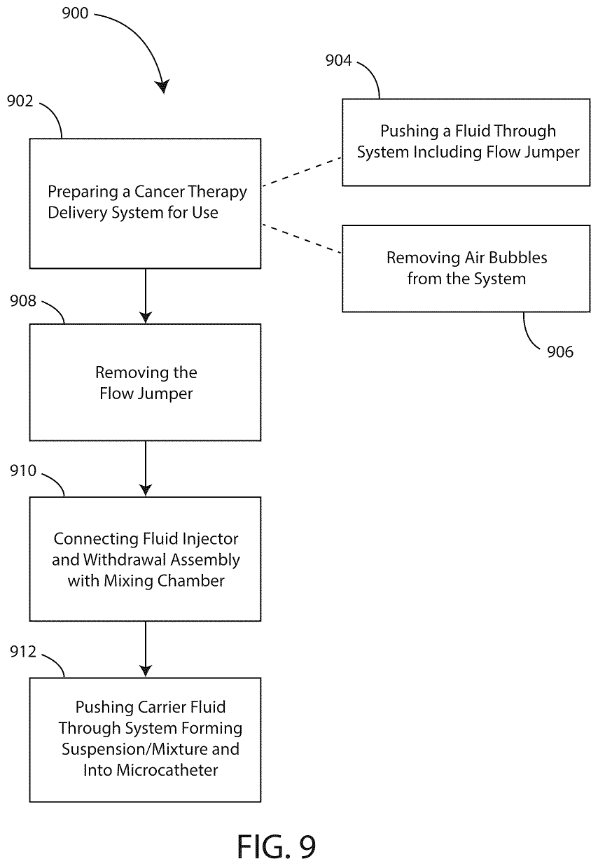

Referring now to FIG. 9, a flow chart of operations of a method of delivering cancer therapy 900 is shown in accordance with various embodiments herein. In an embodiment, such a method can include preparing a cancer therapy delivery system for use 902. Operations associated with preparing the cancer therapy delivery system for use can include, but are not limited to, one or more of priming the cancer therapy delivery system by pushing a fluid 904 (such as a carrier fluid) from a carrier fluid delivery device through a first fluid line, a fluid injector and withdrawal assembly, a flow jumper spanning portions of the fluid injector and withdrawal assembly, and a second fluid line and removing bubbles 906 from the same. Operations of the method can also include removing the flow jumper 908 and connecting the fluid injector and withdrawal assembly with a mixing chamber 910. The method can also include inserting a carrier fluid into the system with the carrier fluid delivery device causing the carrier fluid to move through the system and through the fluid injector and withdrawal assembly into the mixing chamber to form a suspension or mixture of radioactive therapeutic microspheres 912 and the carrier fluid and then passing the same out of the fluid injector and withdrawal assembly and through a fluid line and into a microcatheter and then into a patient.

In an embodiment of the method, the flow jumper is configured to provide a direct fluid flow path between an inflow conduit and an outflow conduit of the fluid injector and withdrawal assembly.

In an embodiment of the method, the radioactive therapeutic microspheres used have an average diameter of from about 20 μm to about 30 μm. In an embodiment of the method, the radioactive therapeutic microspheres comprise a glass including yttria, alumina, and silica. In an embodiment of the method, the radioactive therapeutic microspheres comprise a Y-90 radioisotope emitting beta radiation.

In an embodiment of the method, cancer therapy is delivered to treat a brain tumor. In an embodiment of the method, cancer therapy is delivered to treat glioblastoma.

However, treatment of other types of tumors is also contemplated herein. By way of example, tumors and/or tissue of the head or neck, liver, breast, cervix, prostate, or eye, as well as other tissues can be treated in accordance with embodiments herein. In some embodiments it can be used to treat non-cancerous tumors or other tissue.

### Microspheres

Microspheres herein can include those with a combination of yttria, alumina, and silica. By way of example, in some embodiments, microspheres herein can include Y₂O₃—Al₂O₃—SiO₂ in a 40:20:40 wt. % ratio. It will be appreciated however, that other types of microspheres are also contemplated herein.

In some embodiments, microspheres can be prepared by combining yittrium-89 with alumina and silica, in some cases also using a flame spheroidization method, and using neutron bombardment to convert Y-89 into the beta emitting radioisotope Y-90. In various embodiments, the amount of beta radiation can exceed 2500, 3000, 4000, 5000, 6000, 7000, 8000, or even 9000 Bq per sphere at the time of activity calibration (recognizing that the amount of radiation will drop after that point as the Y-90 radioisotope decays). In some embodiments, the microspheres can be provided in a vial with activity of 3 GBq or lower up to 20 GB q or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, or 0.1 GBq, or less at calibration time, or an amount falling within a range between any of the foregoing.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gy, 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 20 micrometers (μm) to about 30 μm. However, in some embodiments the microspheres can be somewhat smaller or larger.

The density of the microspheres can be quite high. In some embodiments, the density of the microspheres can be above 3 g/mL, such as from 3.1 to 3.5 g/mL, or about 3.3 g/mL. By comparison, the density of water at room temperature is about 0.9978 g/mL. As such, the density of microspheres is much higher than a saline solution which influences how readily such microspheres can settle out of a suspension.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cancer therapy delivery system comprising:
a carrier fluid delivery device;
a first fluid line, wherein the first fluid line is in fluid communication with the carrier fluid delivery device; and
a fluid injector and withdrawal assembly, the fluid injector and withdrawal assembly comprising
an inflow conduit; and
an outflow conduit;
wherein the fluid injector and withdrawal assembly is in fluid communication with the first fluid line;
a second fluid line, wherein the second fluid line is in fluid communication with the outflow conduit; and
a removable jumper device, wherein the removable jumper device is configured to provide a direct fluid flow path between the inflow conduit and the outflow conduit.

2. The cancer therapy delivery system of claim 1, wherein the carrier fluid delivery device includes a syringe assembly.

3. The cancer therapy delivery system of claim 1,
the inflow conduit comprising a first needle; and
the outflow conduit comprising a second needle.

4. The cancer therapy delivery system of claim 3, wherein the removable jumper device fits over a bottom end of the first needle and a bottom end of the second needle when the needles are in a deployed position.

5. The cancer therapy delivery system of claim 1, the removable jumper device comprising a cap.

6. The cancer therapy delivery system of claim 1, the removable jumper device comprising a cap, wherein the cap is configured to be attached to a bottom end of the fluid injector and withdrawal assembly.

7. The cancer therapy delivery system of claim 6, wherein the cap is configured to be attached to a bottom end of the fluid injector and withdrawal assembly using a pressure-fit mechanism.

8. The cancer therapy delivery system of claim 6, the cap defining an internal volume serving as a flow path.

9. The cancer therapy delivery system of claim 8, wherein the internal volume serving as a flow path has a volume of less than 5 ml.

10. The cancer therapy delivery system of claim 6, the cap defining an internal passageway serving as a flow path.

11. The cancer therapy delivery system of claim 1, further comprising a deployment restriction device;
wherein the deployment restriction device is removably attached to the fluid injector and withdrawal assembly; and
wherein the deployment restriction device is configured to limit vertical travel of the inflow conduit and the outflow conduit.

12. The cancer therapy delivery system of claim 1, further comprising:
a first deployment lever, wherein the first deployment lever is configured to move vertically along the fluid injector and withdrawal assembly causing vertical movement of the inflow conduit; and
a second deployment lever, wherein the second deployment lever is configured to move vertically along the fluid injector and withdrawal assembly causing vertical movement of the outflow conduit.

13. The cancer therapy delivery system of claim 12, wherein the first deployment lever and the second deployment lever are configured to slide vertically within slots defined by a body member of the fluid injector and withdrawal assembly.

14. The cancer therapy delivery system of claim 1, wherein the removable jumper device is configured to be removed before the fluid injector and withdrawal assembly is fitted into a mixing chamber.

15. The cancer therapy delivery system of claim 1, wherein one or more of the outflow conduit and the second fluid line have an inner diameter less than an inner diameter of one or more of the inflow conduit, and the first fluid line.

16. A method of delivering cancer therapy comprising:
preparing a cancer therapy delivery system for use, the preparing for use comprising
pushing a fluid from a carrier fluid delivery device through a first fluid line, a fluid injector and withdrawal assembly, a flow jumper spanning portions of the fluid injector and withdrawal assembly, and a second fluid line; and
removing air bubbles from the system;
removing the flow jumper;
connecting the fluid injector and withdrawal assembly with a mixing chamber; and
pushing a carrier fluid through the system with the carrier fluid delivery device causing the carrier fluid to move through the system and through the fluid injector and withdrawal assembly into the mixing chamber to form a suspension or mixture of radioactive therapeutic microspheres and the carrier fluid and out of the fluid injector and withdrawal assembly and through a fluid line and into a microcatheter.

17. The method of delivering cancer therapy of claim 16, wherein the flow jumper is configured to provide a direct fluid flow path between an inflow conduit and an outflow conduit of the fluid injector and withdrawal assembly.

18. The method of delivering cancer therapy of claim 16, wherein the radioactive therapeutic microspheres have an average diameter of about 20 μm to about 30 μm.

19. The method of delivering cancer therapy of claim 16, wherein the method of delivering cancer therapy is applied to treat a brain tumor.

20. The method of delivering cancer therapy of claim 16, wherein the method of delivering cancer therapy is applied to treat glioblastoma.
